(19) United States
(12) Patent Application Publication
(10) Pub. No.: US 2020/0240263 A1
(43) Pub. Date: Jul. 30, 2020

(54) RUGGEDIZED BUOYANT MEMORY MODULES FOR DATA LOGGING AND DELIVERY SYSTEM USING FLUID FLOW IN OIL AND GAS WELLS

(71) Applicants: CALIFORNIA INSTITUTE OF TECHNOLOGY, Pasadena, CA (US); CHEVRON U.S.A. INC., San Ramon, CA (US)

(72) Inventors: Stewart SHERRIT, LA CRESCENTA (US); Jeffery L. HALL, SOUTH PASADENA, CA (US); Dyung Tien VO, THE WOODLANDS, TX (US); Mark Anthony EMANUELE, PRESCOTT, AZ (US)

(21) Appl. No.: 16/651,221

(22) PCT Filed: Oct. 12, 2018
(86) PCT No.: PCT/US2018/055565

§ 371 Date: Mar. 26, 2020

Related U.S. Application Data
(60) Provisional application No. 62/572,309, filed on Oct. 13, 2017.

(51) Int. Cl. E21B 47/26; G01N 33/28; E21B 47/06; E21B 47/07; E21B 47/10; E21B 49/08; G01V 9/00

(52) U.S. Cl. CPC E21B 47/26; G01N 33/2823; E21B 47/06; G01V 9/00; E21B 47/10; E21B 49/08; E21B 47/07

(57) ABSTRACT

Systems and methods for delivering detailed information about physical properties, including inflow data, in a downhole of a well to the surface without the need of providing cabling to the downhole are presented. Such information can be based on data captured by sensors placed within the downhole of the well, and subsequently stored into memory of ruggedized buoyant memory modules (RBMMs) that are physically injected into the fluid flow of the well. The RBMMs use the flow of the fluid inside of the well to deliver the data to a location where the data can be extracted. Data stored in the RBMMs can be extracted either directly from the RBMMs or remotely via, for example, a wireless interface.

### CROSS REFERENCE TO RELATED APPLICATIONS

The present application claims priority to and the benefit of co-pending U.S. provisional patent application Ser. No. 62/572,309 entitled “Ruggedized Buoyant Memory Modules for Data Logging and Delivery System Using Fluid Flow in Oil Wells (RBMM)”, filed on Oct. 13, 2017, the disclosure of which is incorporated herein by reference in its entirety.

### TECHNICAL FIELD

The present disclosure generally relates to systems and methods for measuring and delivering of data from a downhole of a well. In particular, it relates to buoyant ruggedized memory modules for data logging and delivering system using fluid flow in oil/gas wells.

### BACKGROUND

Detailed information about physical properties, including reservoir inflow, in a downhole of an oil-gas producing well, is important to help optimize production and field development. Inflow data, such as oil-gas-water flow rates, pressure, temperature, etc., are key information that help understand the state of nature of the reservoir properties and the effect of well drilling and completion methods. Although useful, the inflow data are not often measured along the lateral section of the well due to the technical or cost-prohibitive challenges. Instead, surface well-head production data (total flow rates, pressure, temperature, etc.) are measured for well performance diagnostic and reporting purposes.

Attempts to instrument the well with continuous electrical or fiber optic cables for powering sensors to measure and deliver physical properties in the downhole of the well have not been successful and/or have not been cost effective. This is particularly true for modern wells with long laterals and multiple perforations of their casing pipe to contact the rock formation and followed by high pressure hydraulic fracturing to increase hydrocarbon inflows from oil-bearing rock formations, is used. Such harsh activities can easily damage power and data cables if present in the downhole of the well.

Unconventional tight rock geologic formations may require a large number of oil/gas wells (holes) drilled in close proximity to each other to effectively extract the hydrocarbon contained in a field. As shown in FIG. 1, horizontally-drilled wells may be used in these applications since the hydrocarbon-bearing rock formations tend to exist in stratified layers aligned perpendicular to the gravity vector. The vertical section of these wells can be 1-2 km below the surface and can extend laterally (i.e., horizontal direction) for distances of, for example, 2-3 km or even more. Oil, natural gas and water may enter the well at many locations (production intervals/zones open to perforations and fracturing) formed along a lateral distance (e.g., 2-3 km or more) of the well with local flow rates and composition (e.g. oil/water fractions) varying due to inherent geology and the accuracy with which the well intersects (e.g., at the production intervals or sections) the oil-bearing rock formations. In general, information about the performance or hydrocarbon delivery and capacities of the well, such as for example, flow rates, pressures and composition, can only be measured at the surface of the well as combined values and with little or no knowledge of individual contributions from each of the production intervals or zones. Lack of local information of the inflow details of the well, at for example, the production intervals or zones, can be a barrier to improving the efficiency of oil-gas extraction from the overall field.

A person skilled in the art readily knows that better knowledge of local interval inflow data (e.g. physical properties such as flow rates, pressure, temperature, etc.) at the downhole of the well (e.g., along the horizontal lateral/section of the well) may help in making better decisions about placement of subsequent perforation/completion intervals for production in a well and/or subsequent drilling of other wells in the field, such as that shown in FIG. 1.

For example, with reference to FIG. 1, an oil production field having a variety of drilled wells, including an unconventional horizontal oil well that extracts oil from shale and tight formation through a plurality of production intervals or zones (shown as rectangles), is shown. In order to develop the field, producing the hydrocarbon-bearing rock formations, a number of wells (i.e., holes) that are spaced, for example in the order of 300 feet apart from each other, may be drilled. These wells are drilled and completed serially, so information that may be gathered from a downhole of, for example, a first well, can aid in determining where to perforate the casing and apply hydraulic fracturing selected intervals of the formation in a second and following wells.

The teachings according to the present disclosure solve the above problems associated with cabling of a downhole of a well while providing detailed information about local physical properties, including inflow data, in the downhole, that can be used for optimizing production and drilling of subsequent wells.

### SUMMARY

The present disclosure describes systems and methods for delivering detailed information about physical properties, including inflow data, in a downhole of a well to the surface without the need of providing cabling to the downhole. Such information can be based on data captured by sensors placed within the downhole of the well, and subsequently stored into memory of ruggedized buoyant memory modules (RBMM) according to the present disclosure that are physically injected into the stream of fluid flow downhole of the well. The RBMMs use the flow of the fluid inside of the well to deliver the data to a location where the data can be extracted. Data stored in the RBMMs can be extracted either directly from the RBMMs or remotely via, for example, a wireless interface.

Although the present systems and methods are described with reference to wells used in the oil industry, such systems and methods may equally apply to other industries, such as, for example, deep sea exploration to send data from underwater robotic vehicles without the need of said vehicles to surface and transmit the data; or through-ice exploration to get data gathered from melt probes by floating the RBMMs up through a melt probe hole while the melt probe hole remains open, or by tying the RBMMs to respective radioactive heating units RHUs to melt their way up from under the ice while the melt probe hole is closed.

According to one embodiment the present disclosure, a system for delivering information about physical properties in a downhole of a well is presented, the system comprising: an autonomous data collection and injection center (DCIC) at a location in the downhole, the DCIC comprising: one or more sensors configured to sense the physical properties at the location in the downhole; and one or more ruggedized buoyant memory modules (RBMMs), each configured to float in a fluid of the well, wherein the DCIC is configured to write data corresponding to sensed physical properties by the one or more sensors into an RBMM of the one or more RBMMs and injects said RBMM into the fluid for conduction of the RBMM by a flow of the fluid to a location for readout of the data.

According to a second embodiment of the present disclosure, a method for delivering information about physical properties in a downhole of a well is presented, the method comprising: i) positioning an autonomous data collection and injection center (DCIC) at a location in the downhole, the DCIC comprising: one or more sensors configured to sense the physical properties at the location in the downhole; and one or more ruggedized buoyant memory modules (RBMMs), each configured to float in a fluid of the well, ii) sensing via the one or more sensors the physical properties at the location in the downhole; iii) based on the sensing, writing data corresponding to sensed physical properties into an RBMM of the one or more RBMMs; and iv) injecting the RBMM into the fluid for conduction of the RBMM by a flow of the fluid to a location for readout of the data.

Further aspects of the disclosure are shown in the specification, drawings and claims of the present application.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 illustrates a cross sectional view of an exemplary known oil production field, comprising one or more drilled wells for production of oil and/or gas.

FIG. 2 shows an exemplary embodiment according to the present disclosure of a data collection and injection center (DCIC) that is positioned in a downhole of a well of the oil production field shown in FIG. 1.

FIG. 3 shows an exploded view of an RBMM according to an exemplary embodiment of the present disclosure.

FIG. 4A shows a picture of an exemplary embodiment of an actual RBMM according to the present disclosure having a substantially spherical enclosure, wherein the enclosure top of the RBMM is removed.

FIG. 4B shows a picture of the RBMM of FIG. 4A in a closed state wherein the enclosure top and bottom are mated.

FIG. 5A shows a diagram of an exemplary embodiment according to the present disclosure, wherein data of an RBMM injected by the DCIC of FIG. 2 is read at the surface of the well.

FIG. 5B shows a diagram of an exemplary embodiment according to the present disclosure, wherein data of an RBMM injected by the DCIC of FIG. 2 is read at a location of the well and transferred though wire to the surface of the well.

FIG. 5C shows a diagram of an exemplary embodiment according to the present disclosure, wherein data of an RBMM injected by the DCIC of FIG. 2 is read at a location of the well and transferred to a relay RBMM that is read at the surface of the well.

FIG. 6 shows an exemplary embodiment according to the present disclosure of an RBMM relay center that reads data from the RBMM injected by the DCIC of FIG. 2 and transfers the data to the relay RBMM of FIG. 5C.

### DEFINITIONS

As used herein the term “ruggedized” may refer to a device or system that is specifically designed to reliably operate in harsh environments and conditions, such as, for example, corrosive and/or erosive environments with high temperatures, pressures and vibrations that may be present in a downhole of well, either during drilling or production of the well. As known in the art, generally ruggedization of a device may include provision of a case of the device that is specifically designed in view of the harsh environments and conditions to protect components and/or systems internal to the case. Furthermore, such components and/or systems may be designed with increased tolerance to the harsh environments and conditions.

As used herein the term “buoyant” may refer to the property of an object to float when immersed in a fluid. In other words, an upward force exerted by the fluid on the object opposes the weight of the immersed object.

As used herein the expression “memory module” may refer to a device that comprises a memory for data storage and retrieval.

As used herein the term “autonomous” may refer to a device or system that is self-sufficient in performing tasks for which is was designed. Accordingly, such autonomous device or system may include a local power source.

### DETAILED DESCRIPTION

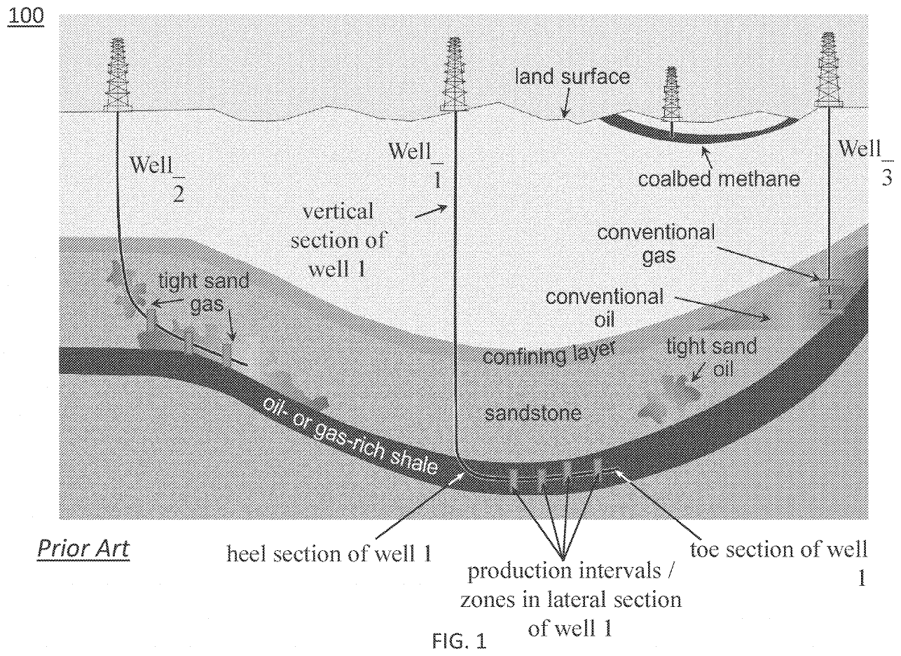

FIG. 1 illustrates a cross sectional view of an exemplary oil production field 100, comprising one or more drilled wells (Well_1, Well_2, . . . ) for production and extraction of oil and/or gas from various regions of the field. A person skilled in the art is well aware of items of the production field 100 indicated in FIG. 1, the description of which is beyond the scope of the present disclosure. In particular, as can be seen in FIG. 1, a vertical section of the Well_1 may be drilled to reach and penetrate an oil- or gas-rich shale (e.g., rock formation), and a lateral (e.g., horizontal) section of the Well_1, which in the exemplary case of FIG. 1 is substantially horizontal, may be drilled along the shale, starting from a heel section of the Well_1, and ending at a toe section of the Well_1. A person skilled in the art would know that the vertical section of the Well_1 may extend 1 to 2 km below the surface and the lateral section of the Well_1 may extend for distances of, for example, 2-3 km or more.

With continued reference to FIG. 1, as it is well known by a person skilled in the art, fluids, including oil, water, and natural gas, may enter the Well_1, for example through a casing of the Well_1, at production perforated intervals/zones that may be formed in the lateral section of the Well_1. Each of such production intervals/zones may include holes and/or openings that extract the fluid from the shale and route into (the casing of) the Well_1. As shown in FIG. 1, the perforated intervals/production zones may be separated by distances of, for example, about 100 meters (i.e., about 300 feet), and between each of the interval (or stage) there are several clusters of perforations with closer spacing in order to cover a lengthy lateral and extract more hydrocarbon from shale/tight formations. Since there are many production zones, the inflow contribution for each of the interval or zone, such as, for example, local pressure, temperature, flow rates and composition, may vary due to inherent geology and the accuracy with which the lateral section of the Well_1 intersects the oil-bearing rock formations at the production zones.

As described above, collecting data at regions of the Well_1, for example close to each of the production zones, can help evaluate effectiveness of each of the production zones and further help in optimizing production. Systems and methods according to the present disclosure collect data from battery powered sensors that are placed inside of a well, including data related to, for example, pressure, temperature, flow rates and composition (e.g., fraction of oil, gas, water). Such data collected by the sensors can subsequently be logged, for example as a function of time, and saved to the ruggedized buoyant memory module (RBMM) according to the present disclosure. In turn, each of the RBMMs may be injected into the flow of the fluid and extracted at the top of the well (e.g., Well_1 of FIG. 1), or at a location close to the heel of the well, for reading by an operator or a computer.

According to an embodiment of the present disclosure, timing between the injection of each of the RBMMs can be adjusted according to any desired scheme. For example, it may be desirable to provide more data updates, and therefore higher frequency of injection of the RBMMs, in an early stage of a production zone where a change in local physical properties, such as, for example, flow, pressure, etc., may be high, and provided less data updates, therefore lower frequency of injection, in later stages of the production zone.

The systems and methods according to the present disclosure circumvent problems related to cabling in the downhole of the well by using the flow of fluid inside of the well to physically deliver the data. According to an embodiment of the present disclosure, sensors placed at each production zone of a well may be used to collect and send data over a set (e.g., fixed) interval to aid in determining the production efficiency of each production zone. Furthermore, data collected from one well (e.g., from all the production zones) can help in determining position and other related production parameters of subsequent wells.

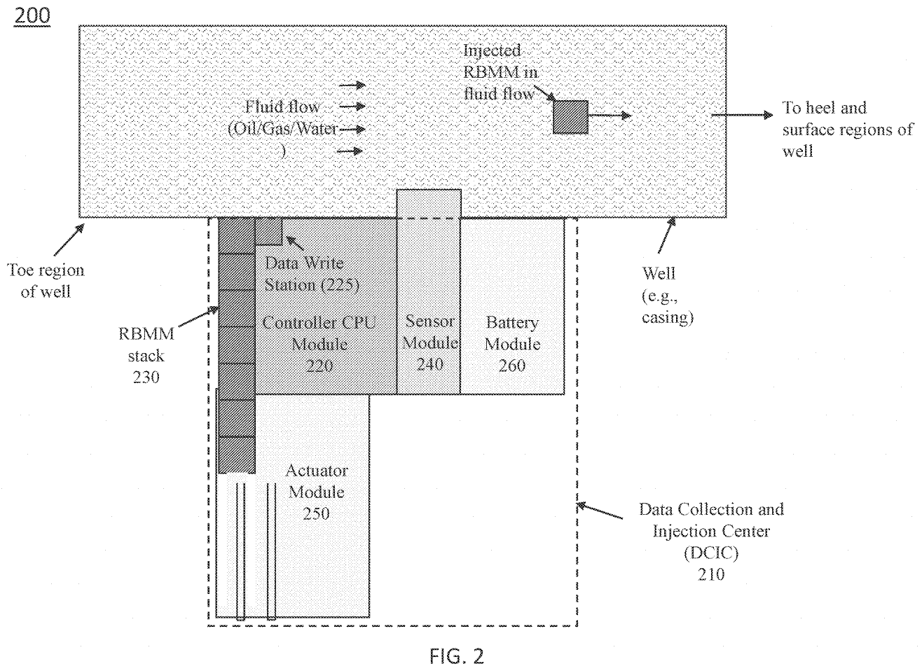

FIG. 2 shows an exemplary embodiment according to the present disclosure of a self-contained and autonomous data collection and injection center DCIC (210) that is positioned (e.g., fixated or placed) in a downhole of a well of the oil production field shown in FIG. 1. As described above, one or more of such DCIC (210) may be positioned at different locations of the downhole of the well. Such locations may be at or near production zones of the well. As shown in FIG. 2, the DCIC (210) may include a plurality of RBMMs.

It should be noted that methods and devices for placement of components inside of the downhole are well known by a person skilled in the art and not the subject of the present disclosure when referred to a DCIC placement. One or more DCIC (210) may be placed at various locations of the downhole from which local information may be desired. Such locations may include production zones formed inside the well from which oil, gas, and/or water may enter the well. Furthermore, it should be noted that systems and methods according to the present teachings may apply to any downhole containing fluids, whether a conventional vertical downhole, or unconventional horizontal (lateral) downhole (e.g., as known in fluid extraction via hydraulic fracturing), and irrespective of presence of a casing within the downhole.

The DCIC (210) according to the present disclosure is an autonomous device that is powered by a battery module (260). The battery module may provide powering to various elements of the DCIC (210). The battery module may have enough charge to power the DCIC (210) through the life of the DCIC (210) when positioned in the downhole.

With further reference to FIG. 2, the DCIC (210) according to the present disclosure may include a sensor module (240) that includes sensors for gathering data. Such sensors may be exposed (e.g., in contact) with the inside region of the well, inclusive of the fluid, so to sense relevant local physical properties of the well, such as, for example, flow rate, composition, temperature and pressure. The DCIC (210) may be encased within a ruggedized enclosure that protects the DCIC (210) from harsh local downhole environment while providing adequate exposure of the sensors to the environment.

The DCIC (210) of FIG. 2 may include a central processing unit CPU module (220) to control operation of the DCIC (210), including, but not limited to, control of the sensors, data read from the sensors, storage and manipulation of data read from the sensors, date and time (e.g., clock) generation, data write to the RBMMs, and injection of the RBMMs into the fluid flow. A person skilled in the art would know of many different design and implementations of the CPU module (220) which are beyond the scope of the present application. It should be noted, however, that such CPU module (220) may be based on readily available off-the-shelf devices and/or proprietary designs using well known in the art methods and tools, which in combination allow implementation of a cost-effective solution for gathering of downhole information. The CPU module (220) may include a memory, both volatile and non-volatile, for executing stored programs and storing data sensed from the sensor module (240).

The DCIC (210) of FIG. 2 may include a stacking module (230) for arranging the RBMMs in a stacked configuration in preparation for writing data to, and injecting into the fluid flow, each of the RBMMs. An actuator module (250) actuates, under control of the CPU module (220), the stacking module (230) for injecting (e.g., dispensing) one of the RBMMs into the fluid flow.

With continued reference to FIG. 2, the DCIC (210) may include a plurality of RBMMs which can be injected into the fluid flow periodically. The frequency of injection of the RBMMs into the fluid flow may be pre-programmed into a memory of the DCIC (e.g., within the CPU) according to a lookup table or a formula that is a function of one or more parameters, including time, date, and any of the collected (e.g., sensed) local physical properties. A person skilled in the art is well aware that during production, information from the downhole of the well may be desired at a higher frequency during a beginning phase of the production, and lower frequency during latter phases of the production. Accordingly, any desired frequency of injection of the RBMMs may be pre-programmed into the memory of the DCIC (210). A number of the RBMMs modules included in each DCIC (210) may the adjusted, prior to placement in the downhole of the DCIC (210), according to the desired frequency of injection and life of the DCIC (210). According to some exemplary embodiments, tens to hundreds of such RBMMs may be included in each DCIC (210).

Once the DCIC (210) determines, for example, via a program executed by the CPU module (220), that it is time to inject data (e.g., one RBMM) into the fluid flow, the CPU module (220) may write data, via a data write station (225), into an RBMM at the top of the stacking module (230) and actuate the actuator module (250) to inject the RBMM into the fluid flow of the well. Data written into the memory of the RBMM may include data from the sensor module (240), along with time stamp and a unique identification code that uniquely identifies the RBMM, based on, for example, a corresponding DCIC (210), placement along the well (e.g., production zone) and/or placement along the stacking module (230). It should be noted that the data write station (225) may write data into a memory of the RBMM based on any known in the art physical and logical interface, including via physical wires or wirelessly. As noted above, the elements of the DCIC (210), including elements of (220, 225, 230, 240, 250, and 260) may be based on readily available off-the-shelf devices for a cost-effective implementation.

As shown in FIG. 2, once injected, and in view of the buoyancy of the (injected) RBMM, the RBMM is conducted by the fluid flow through the well and in a same direction as the fluid flow, to a location of the well where the RBMM can be read, collected, or further manipulated. Such location may be the surface of the well, or for example, a heel of the well. Due to the harsh environment within the downhole of the well, the RBMM is designed to withstand temperatures above 85° C., and up to, for example, 125° C.

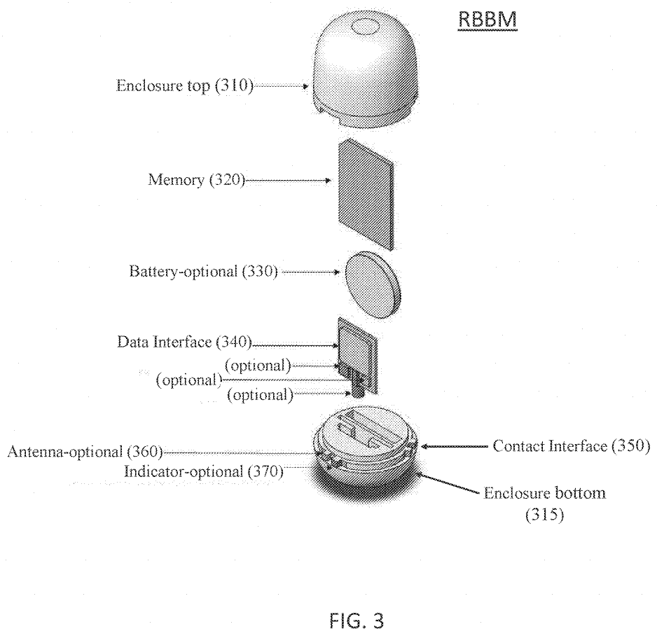

FIG. 3 shows an exploded view of an RBMM according to an embodiment of the present disclosure. As shown in FIG. 3, the RBMM according to the present disclosure comprises an enclosure top (310) that is mated with an enclosure bottom (315) to encapsulate, in a ruggedized fashion, at least one memory device (320). A data interface board (340) may be used to write and/or read data to/from the memory device (320) via, for example, a contact interface (350). An optional battery (330) may be provided to power the RBMM, such as, for example, allow autonomous readout of the data from the memory device (320), and/or, allow wireless communication via, for example, an optional antenna (360). An optional indicator (370), such as for example, a light-emitting diode (LED), may be used to help in locating the RBMM once at the surface of the well.

With continued reference to FIG. 3, according to an exemplary embodiment of the present disclosure, the data interface board (320) may be simple conductor traces that electrically connect the memory device (320) to electrodes of the contact interface (350). Such exemplary embodiment may allow implementation of the RBMM as a passive device with no requirement for a local power source (battery) within the RBMM. Accordingly, such passive RBMM may minimally include the memory device (320) with means to write to the memory device (320) when stacked in the stacking module (230) prior to injection into the fluid flow. Such means may be, for example, a simplified data interface board (340) and/or the contact interface (350). Furthermore, according to some exemplary embodiment of the present disclosure, such passive RBMM may be fitted with passive radio frequency identification (RFID) tags for identification and localization of the RBMM. In turn a reader may use such passive RFID to locate/identify the RBMM prior to reading the data. According to other exemplary embodiments of the present disclosure, such passive RBMM may be configured for inductive coupling of data and/or power via methods and devices that are well known in the art.

With further reference to the RBMM of FIG. 3, the enclosure top (310) and bottom (315) may be made of any material known in the art that may protect (shield) the memory device (320) and other elements (optional) encased within the enclosure in view of known downhole conditions (e.g., temperature, pressure, flow rate, composition), while providing sufficient buoyancy for a small volume of the RBMM. Various metals, such as stainless steel and titanium, and various polymers may fit such requirements.

According to an exemplary embodiment of the present disclosure, a shape of the RBMM, as dictated by a shape of the enclosure top (310) and bottom (315) when mated, can be substantially spherical as shown in FIG. 3, or substantially bullet shaped. Other three-dimensional shapes, including shapes with either rounded or squared edges may also be envisioned. It should be noted that a shape of the RBMM may not be considered as critical per se and may rather be a function of other design goals and requirements, such as, for example, stacking of the RBMMs within the DCIC (210) and/or RBMMs collecting methods and devices used at the surface of the well or other collection locations.

Data stored in the RBMM can be extracted by any means known in the art. According to an exemplary embodiment of the present disclosure, such data can be extracted via a manual means, wherein the RBMM is first located and then physically handled (e.g., human or robotic arms) to combine an element of the RBMM, such as for example, the memory device (320) (e.g., a solid-state memory device), into a reading station that extracts (reads) the data stored into the memory device (320).

According to another exemplary embodiment of the present disclosure, the data stored in the RBMM can be extracted via autonomous means, wherein the RBMM is first located and then physically handled (e.g., human or robotic arms) to read the data directly from the RBMM via, for example, an integrated interface/reader (340) of the RBMM. An optional integrated indicator (370) (e.g., LED) may help in localizing the RBMM, or alternatively, localization and identification of the RBMM may be provided via passive RFID tagging as described above. In this exemplary case, data read from the RBMM may be provided via a small battery (330) integrated within the RBMM. Such battery (330) may be a rechargeable battery that is charged prior to data storage into the RBMM and injection of the RBMM into the fluid flow. A person skilled in the art is well aware of other means for provision of power to the RBMM, such as, for example, radiated power that may be used to charge power storage cells (e.g., capacitor banks) within the RBMM prior to either writing or reading data into the memory device (330) (e.g., solid-state memory) of the RBMM.

According to yet another exemplary embodiment of the present disclosure, the data stored in the RBMM can be extracted via remote/wireless means. In such embodiment, data from the RBMM can be read wirelessly, for example through an integrated antenna (360), without the need to (precisely) locate and physically handle the RBMM. Power for remote/wireless transmission of the data stored in the RBMM may be provided via a small battery (330) integrated in the RBMM. Such battery (330) may be a rechargeable battery that is charged prior to data storage into the RBMM and injection of the RBMM into the fluid flow. Alternative methods for provision of power to the RBMM as described above may also be considered by a person skilled in the art.

With further reference to FIG. 3, the memory device (320) may be a solid-state memory device. A person skilled in the art is well aware of many types of solid-state memory devices that use, for example, electrically-programmable non-volatile flash memory, that can maintain stored data without need for external power. Some such devices include, for example, MMC, SD, SSD, USB flash drive, and SIM cards. According to an exemplary embodiment of the present disclosure, the RBMM may use a non-volatile flash memory and be a passive device with no battery and/or local power provided within the RBMM. According to another exemplary embodiment of the present disclosure, the RBMM may include a battery, or a rechargeable battery, and transmit data remotely or wirelessly.

According to an exemplary embodiment of the present disclosure, the size of the memory device (320) of the RBMM may be sufficient to store one data set from the sensor module (240). In other words, data corresponding to one given time stamp. Accordingly, each RBMM may store one snapshot of the downhole local conditions and therefore, progression of the downhole local conditions in time may be restored by stitching together data from different RBMMs injected at different time stamps. Alternatively, or in addition, the size of the memory of the RBMM may be sufficient to store not only data related to a current time stamp, but also to store data related to previous time stamps (via previously injected RBMMs). This allows provision of historical data related to downhole conditions based on a single RBMMs, and therefore mitigate, via redundancy of data sets, loss of data due to for example to loss of RBMMs injected within the fluid flow.

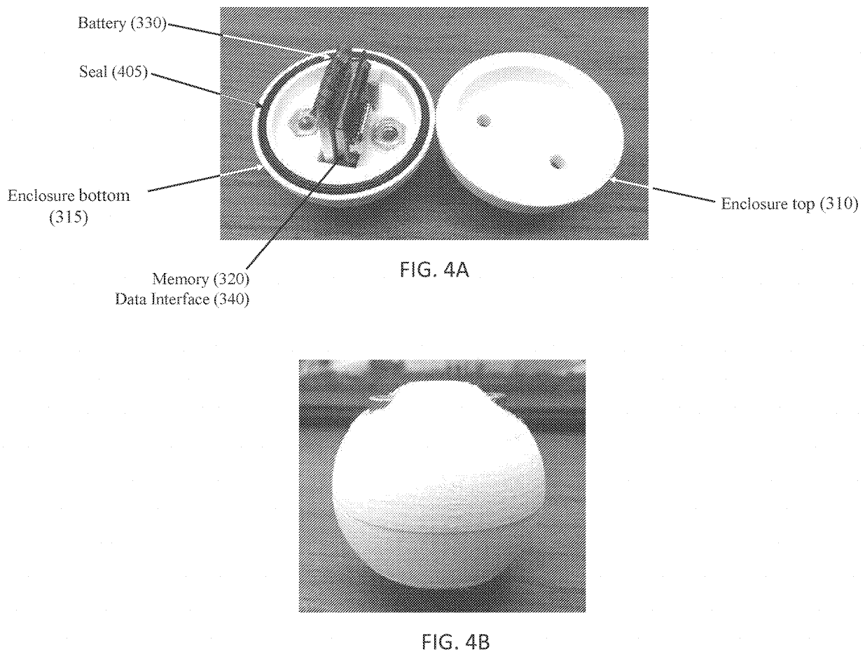

FIG. 4A shows a picture of an exemplary embodiment of an actual RBMM having a substantially spherical enclosure, wherein the enclosure top (310) of the RBMM is removed. In the exemplary embodiment depicted in FIG. 4A, the RBMM comprises the memory device (320), the battery (330), the data interface (340) described above with reference to FIG. 3, encapsulated within the enclosure top (310) and the enclosure bottom (315) that may be sealed via a seal (e.g., gasket, rubber washer) (405). As can be seen in FIG. 4A, according to an exemplary embodiment of the present disclosure, the elements (320, 330, 340) are fitted within slots formed in the enclosure bottom (315). A person skilled in the art would know of many methods for fabricating the enclosure bottom (315) and top (310), including for example, molding methods. According to an exemplary embodiment of the present disclosure, a diameter of the RBMM in a closed state, as shown in FIG. 4B, may be about 2 centimeters or less. A person skilled in the art would realize that the RBMM according to the present teachings is not limited to a specific size, as a desired buoyancy of the RBMM may be achieved for any size of the RBMM.

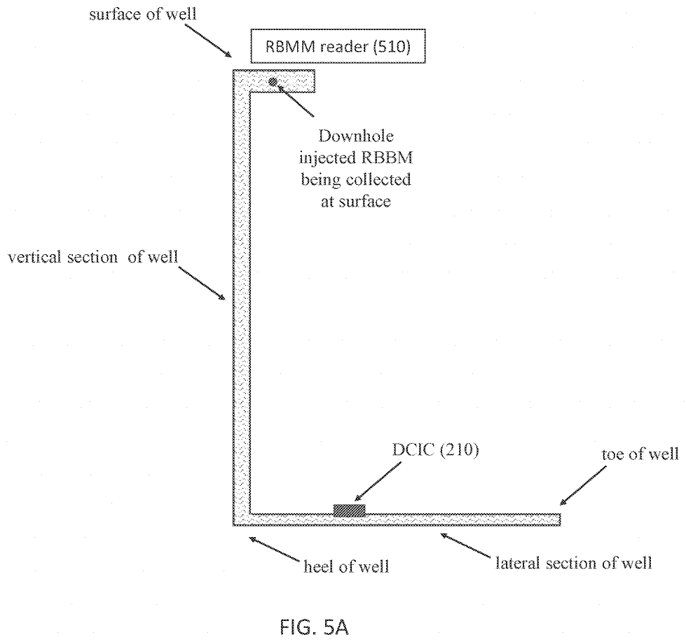

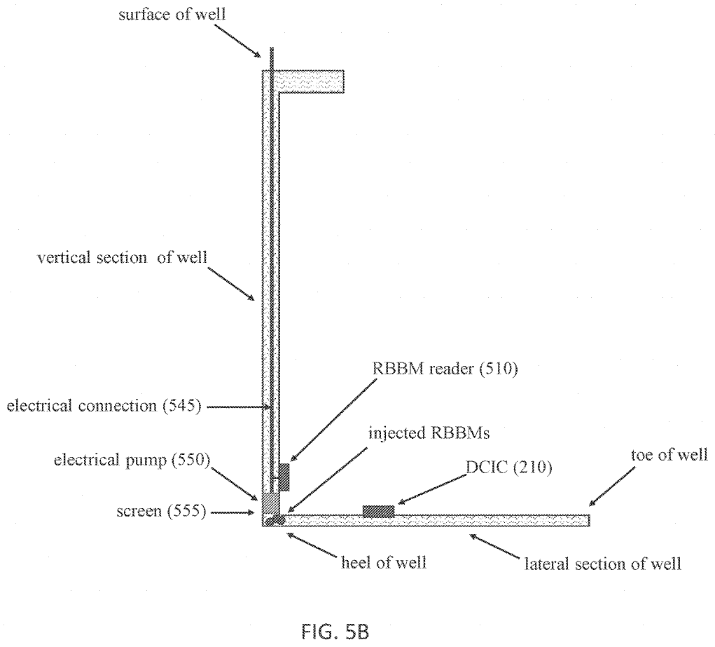

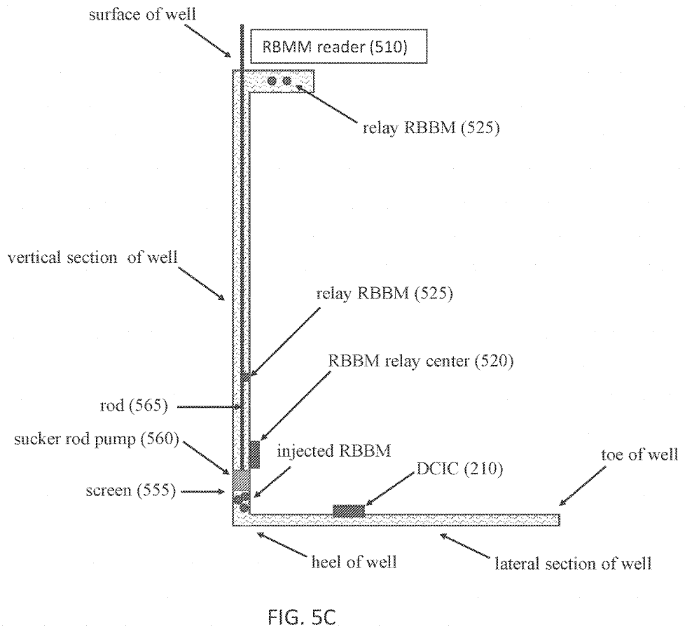

FIG. 5A shows a diagram of an exemplary embodiment according to the present disclosure, wherein a DCIC (210) is used in a downhole of a well to gather data corresponding to local properties of the downhole. In the exemplary embodiment depicted in FIG. 5A, an RBMM that is injected by the DCIC (210) floats to the surface of the well where an RBMM reader (510) can extract data stored in the RBMM. As described above, such data may be extracted (read) by any of a manual, autonomous, or remote/wireless means.

With continued reference to the diagram of FIG. 5A, as described above, floating of the injected RBMM to the surface of the well is provided by a flow of the fluid within the well and a buoyancy of the RBMM in view of known characteristics of the fluid. A person skilled in the art would clearly understand that the flow of the fluid within the well may change as a function of a production phase of the well. For example, in early production stages the flow may be significantly higher than in later production stages. Accordingly, as it is well known in the art, artificial means may be added to the well for extraction (lift) of the fluid (e.g., oil, gas, water) within the well. The exemplary embodiment depicted in FIG. 5A may equally apply during any of the production phases wherein (physical) floating of the RBMM to the surface of the well is not impeded. This includes a production phase where a gas is injected in the downhole (e.g., gas lift) to artificially increase fluid flow.

In some cases, the artificial means for lifting of the fluid within the well may require introduction of a screen (e.g., filter) in a vertical region of the well near the heel of the well, which screen may impede progression/flow of the RBMM to the surface of the well. Two such exemplary cases are shown in FIG. 5B and FIG. 5C, where a screen (555) positioned at a region of the vertical section of the well near the heel of the well impedes progression of the RBMMs towards the surface of the well. The screen may filter larger particles in the fluid to protect a pump that is used to artificially lift the (filtered) fluid. As noted in FIG. 5B, according to an exemplary embodiment, the pump may be an electrical pump (550) that is powered via an electrical connection (545) guided through the vertical section of the well. As noted in FIG. 5C, according to an exemplary embodiment, the pump may be a sucker rod pump (560) that is (mechanically) powered via a rod connection (565) guided through the vertical section of the well.

As shown in FIGS. 5B and 5C, the screen (555) may impede progression of the RBMMs injected by DCIC (210). It follows that according to an embodiment of the present disclosure, extraction of the data from the RBMM may be performed in a location in the downhole that is away from the DCIC (210), such as, for example, by the screen (555) positioned near the heel of the well.

With further reference to FIG. 5B, a diagram of an embodiment according to present disclosure is shown, wherein an RBMM reader (510) is placed in the downhole of the well on a side of the screen (555) away from the injected (and entrapped) RBMMs. The reader (510) may remotely/wirelessly read data from the RBMMs, which are positioned at close proximity of the reader (510) and transfer the read data to the surface of well via wires of the electrical connection (545). Such exemplary embodiment according to present disclosure may be used in cases where the screen (555) impedes progression of the injected RBMMs towards the surface of the well and where presence of the RBMMs between the pump (550) and the surface of the well may interfere with production requirements. Accordingly, no RBMM may flow at the surface of the well.

With further reference to FIG. 5C, a diagram of an embodiment according to present disclosure is shown, wherein an RBMM relay center (520) is placed in the downhole of the well on a side of the screen (555) away from the injected (and entrapped) RBMMs. The RBMM relay center (520) may remotely/wirelessly read data from the injected (and entrapped) RBMMs, which are positioned at close proximity of the RBMM relay center (520) and transfer the read data to the surface of well via relay RBMMs (525) that float to the surface of the well. In turn, an RBMM reader (510) at the surface of the well reads the data from the relay RBMMs (525) in a fashion similar to one described with reference to FIG. 5A. Such exemplary embodiment according to present disclosure may be used in cases where a screen (555) impedes progression of the injected RBMMs and where presence of the RBMMs between the pump (550) and the surface of the well may not interfere with production requirements. Accordingly, an injected relay RBMM may flow at the surface of the well.

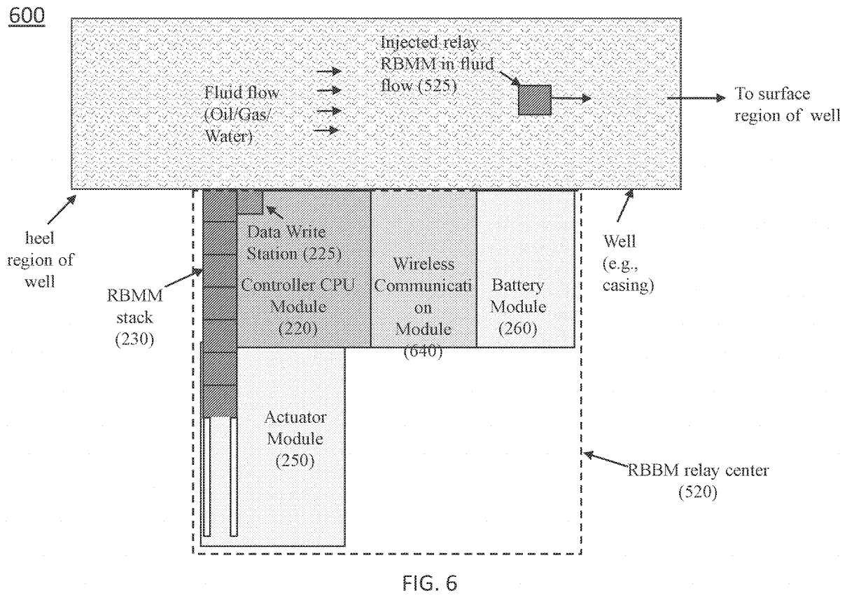

FIG. 6 shows further details, according to an exemplary embodiment of the present disclosure, of the RBMM relay center (520) of FIG. 5C. As can be clearly understood by a person skilled in the art, the RBMM relay center (520) may include the same internal modules (220, 225, 230, 250, 260) as the DCIC (210) of FIG. 2, with the addition of a wireless communication module (640) that can be used to wirelessly read data from the injected (and entrapped) RBMMs below the screen (555). It should be noted that although the RBMMs injected by the DCIC (210) of FIG. 5C may require support of wireless communication for communication with the RBMM relay center (520), the relay RBMMs (525) injected by the RBMM relay center (520) may be simple passive devices as described above, or if desired, active devices with, for example, wireless communication support. The type (passive or active) of the relay RBMMs (525) is solely a function of the RBMM reader (510) located at the surface of the well.

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art as a complete disclosure and description of how to make and use the embodiments of the disclosure and are not intended to limit the scope of what the inventor/inventors regard as their disclosure.

Modifications of the above-described modes for carrying out the methods and systems herein disclosed that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or systems, which can, of course, vary. It is also to be understood that the terminology used herein is for the purpose of describing particular embodiments only and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

1. A system for delivering information about physical properties in a downhole of a well, the system comprising:
an autonomous data collection and injection center (DCIC) at a location in the downhole, the DCIC comprising:
one or more sensors configured to sense the physical properties at the location in the downhole; and
one or more ruggedized buoyant memory modules (RBMMs), each configured to float in a fluid of the well,
wherein the DCIC is configured to write data corresponding to sensed physical properties by the one or more sensors into an RBMM of the one or more RBMMs and injects said RBMM into the fluid for conduction of the RBMM by a flow of the fluid to a location for readout of the data.

2. The system according to claim 1, wherein the one or more sensors comprise one or more of: a) a pressure sensor, b) a temperature sensor, c) a flow sensor, and d) a composition sensor.

3. The system according to claim 1, wherein the location of the DCIC in the downhole is within a lateral section of the well, between a toe and a heel of the well.

4. The system according to claim 1, wherein the location of the DCIC in the downhole is a location near a production zone of the well.

5. The system according to claim 4, further comprising one or more additional DCIC at locations of the well near respective one or more production zones.

6. The system according to claim 1, wherein each of the one or more RBMMs comprises an electrically-programmable non-volatile flash memory device.

7. The system according to claim 6, wherein the electrically-programmable non-volatile flash memory device is one of: a) an MMC card, b) an SD card, c) a SIM card, d) an SSD, and d) a USB flash drive.

8. The system according to claim 1, wherein each of the one or more RBMMs is a passive device devoid of a local power source.

9. The system according to claim 1, wherein each of the one or more RBMMs is an active device comprising a local power source.

10. The system according to claim 9, wherein the local power source comprises one of a battery and a rechargeable battery.

11. The system according to claim 1, wherein each of the one or more RBMMs has a shape that is substantially spherical.

12. The system according to claim 11, wherein the substantially spherical shape has a diameter that is about two centimeters or less.

13. The system according to claim 12, wherein a number of the one or more RBMMs is in a range of 10's to 100's.

14. The system according to claim 1, wherein the location for readout of the data is at a surface of the well.

15. The system according to claim 14, further comprising an RBMM reader at the surface of the well configured to read the data wirelessly.

16. The system according to claim 15, wherein the RBMM reader locates and identifies the RBMM at the surface of the well via passive RFID tagging.

17. The system according to claim 1, wherein the location for readout of the data is at a heel of the well.

18. The system according to claim 17, further comprising an RBMM reader in a vertical section of the well near the heel of the well,
wherein the RBMM reader is configured to read the data wirelessly and transfer the data via a wired connection to the surface of the well.

19. The system according to claim 1, further comprising an RBMM relay center in a vertical section of the well near the heel of the well,
wherein the RBMM relay center is configured to read the data wirelessly and transfer the data to a relay RBMM that the RBMM relay center injects into the fluid for conduction of the relay RBMM by the flow of the fluid to the surface of the well.

20. A method for delivering information about physical properties in a downhole of a well, the method comprising:
i) positioning an autonomous data collection and injection center (DCIC) at a location in the downhole, the DCIC comprising:
one or more sensors configured to sense the physical properties at the location in the downhole; and
one or more ruggedized buoyant memory modules (RBMMs), each configured to float in a fluid of the well,
ii) sensing via the one or more sensors the physical properties at the location in the downhole;
iii) based on the sensing, writing data corresponding to sensed physical properties into an RBMM of the one or more RBMMs; and
iv) injecting the RBMM into the fluid for conduction of the RBMM by a flow of the fluid to a location for readout of the data.

21. A method for optimizing oil production in an oil field, the method comprising:
drilling a first well;
positioning in the vicinity of production zones formed in a lateral section of the first well, one or more of the system for delivering information about physical properties in a downhole of the well according to claim 1;
based on the positioning, gathering information from the production zones; and
based on the gathering, drilling a second well proximate the first well having production zones for optimized production of oil.
